(19) United States
(12) Patent Application Publication
(10) Pub. No.: US 2021/0401670 A1
(43) Pub. Date: Dec. 30, 2021

(54) INTRAVENOUS FLUID CONTAINER VOLUME MONITORING SYSTEM

(71) Applicant: CareFusion 303, Inc., San Diego, CA (US)

(72) Inventors: Daniel M. ABAL, San Diego, CA (US); Brendan John BURGESS, Poway, CA (US); Ramkumar SUBRAMANIAN, San Diego, CA (US)

(21) Appl. No.: 17/359,400

(22) Filed: Jun. 25, 2021

Related U.S. Application Data
(60) Provisional application No. 63/046,544, filed on Jun. 30, 2020.

(51) Int. Cl. A61J 1/18; A61M 5/172

(52) U.S. Cl. CPC A61J 1/18; A61M 5/172; A61J 2200/76; A61M 2205/18; A61M 2205/3389; A61M 2205/6054; A61J 2205/60

(57) ABSTRACT

A system, method and device for determining a volume of a fluid in a medication container is disclosed. An infusion of a medication is initiated from a medication container. The medication container includes one or more radio frequency identification (RFID) tags affixed along a side of the container. A radio frequency (RF) signal is directed from a reader device toward the RFID tags disposed on the medication container. A signal strength of one or more returned respective RF signals from the one or more RFID tags is detected, the returned RF signals including one or more identifiers for identifying the one or more RFID tags. A threshold signal level for determining a level of fluid within the medication container is determined, and a volume of the fluid is then determined by determining which of the returned respective RF signals has a signal strength satisfying the threshold signal level.

### CROSS REFERENCE TO RELATED APPLICATIONS

This application claims the benefit of U.S. Provisional Application Ser. No. 63/046,544, entitled “INTRAVENOUS FLUID CONTAINER VOLUME MONITORING SYSTEM,” filed on Jun. 30, 2020, the entirety of which is incorporated herein by reference.

### TECHNICAL FIELD

This application relates generally to monitoring a volume of a fluid in a medication container.

### BACKGROUND

Intravenous (IV) infusions are typically run un-attended after a caregiver sets an infusion of a given quantity of medication to run for a specific amount of time. Infusion devices (e.g., infusion pumps) can be configured with a volume to be infused (VTBI). For example, a clinician may enter a rate and duration of an infusion, and the infusion device can generate a VTBI. Alternatively, the clinician may start the infusion with a VTBI. Sometime, the clinician can start with a VTBI. Usually, the infusion pump calculates an estimate of an amount that has been infused and gives an alarm when the prescribed VTBI has been attained, notifying the caregiver to change the IV bag. When a notification is not provided in a timely manner, the infusion process can be interrupted.

### SUMMARY

Under normal operations, some pumps run faster, though still within the tolerance of the pumps' performance specifications. Existing pumps do not measure an actual amount infused as a result of the faster running rate. For example, some infusion pump may only calculate a projected amount to be infused (at the nominal rate) that does not include the actual amount over-infused. In other words, there may be an over-infused amount when the pump exceeds the VTBI by an amount that may still be within the tolerance limit. For example, for a pump infusing at 60 ml/hour for eight hours, if it runs 5% faster, the pump will empty a 500 mL bag approximately 20 minutes earlier than is expected. This can result in air being drawn into the pump. In such a case, the pump will sound an alarm after the air has reached an air-in-line (AIL) sensor. The caregiver may then have to disconnect the set, prime it to remove the air, and then restart the infusion. Such an interruption presents a potential for introducing infections, adding time and work for the caregiver, and adding steps that can introduce errors.

Accordingly, there is a need for methods and systems that monitor a volume of a fluid in IV solution containers so that timely notifications that a container is empty, or is about to become empty, can be provided.

The disclosed subject matter relates to a system, device, and method of determining a volume of a fluid in a medication container. In accordance with some implementations, an infusion of a medication is initiated from a medication container (e.g., an IV solution bag or infusion container). The medication container includes one or more electronic tags affixed along a side of the medication container. For the purpose of this disclosure, the devices, systems and method disclosed herein are described as using radio frequency identification (RFID) tags. However, other tags configured to receive and transmit a signal through a liquid medium may also be used.

A monitoring device for monitoring a volume of a medication container comprises one or more radio frequency (RF) devices providing an RF transmitting source and a RF receiving source; one or more processors; and a non-transitory memory device having instructions thereon that, when executed by the one or more processors, cause the monitoring device to perform operations. According to various implementations, the operations comprise transmitting, via the RF transmitting source, an RF signal toward a plurality of RFID tags disposed on a side of a medication container associated with an infusion device administering a medication from the medication container, wherein the side of the medication container is opposite a side of the medication container nearest the RF transmitting source such that the RF signal passes through the medication container before interacting with the RFID tags; detecting, via the RF receiving source, a signal strength of returned RF signals from the RFID tags, each of the returned RF signals including an identifier identifying a respective RFID tag; determining, based on at least one of the returned identifiers, a threshold signal level associated with detecting a fluid within the medication container; determining a volume of the fluid within the medication container based comparing the signal strength of each returned RF signal with the determined threshold signal level; and providing an electronic indication of the volume. Other aspects include corresponding methods, systems, and computer program products for implementation of the monitoring device and its features.

A disclosed method includes directing a radio frequency (RF) signal, from an RF source, toward the one or more RFID tags disposed on the medication container. The method also includes detecting, using an RF reader, a signal strength of one or more returned respective RF signals from the one or more RFID tags, the returned one or more RF signals including one or more identifiers for identifying the one or more RFID tags. The method includes determining, based on the one or more identifiers, a threshold signal level for determining a level of fluid within the medication container, and determining whether the signal strength of the one or more returned respective RF signals satisfies the determined threshold signal level. In accordance with a determination that the signal strength of the returned RF signal satisfies the threshold signal level, the method includes providing an indication that the fluid within the medication container is at a first volume; and in accordance with a determination that the signal strength does not satisfy the threshold signal level, the method includes providing an indication that the fluid within the medication container is at a second volume.

The disclosed subject matter also relates to a machine-readable medium embodying instructions that, when executed by a machine, allow the machine to perform a method for determining a volume of a fluid in a medication container.

The disclosed subject matter also relates to a system for determining a volume of a fluid in a medication container. The system includes one or more processors and a memory including instructions that, when executed by the one or more processors, cause the one or more processors to perform the steps of the method described herein.

The subject technology provides a system for determining a volume of a fluid in a medication container, including one or more processors and a memory. The memory includes instructions that, when executed by the one or more processors, cause the system to initiate an infusion of a medication from a medication container. The medication container includes one or more radio frequency identification (RFID) tags affixed along a side of the medication container. The system directs a radio frequency (RF) signal, from an RF source, toward the one or more RFID tags disposed on the medication container. The system also detects, using an RF reader, a signal strength of one or more returned respective RF signals from the one or more RFID tags, the returned one or more RF signals including one or more identifiers for identifying the one or more RFID tags. The system determines, based on the one or more identifiers, a threshold signal level for determining a level of fluid within the medication container, and determines whether the signal strength of the one or more returned respective RF signals satisfies the determined threshold signal level. In accordance with a determination that the signal strength of the returned RF signal satisfies the threshold signal level, the system provide an indication that the fluid within the medication container is at a first volume; and in accordance with a determination that the signal strength does not satisfy the threshold signal level, the system provides an indication that the fluid within the medication container is at a second volume. Other aspects include corresponding methods, apparatus, and computer program products for implementation of the corresponding system and its features.

It is understood that other configurations of the subject technology will become readily apparent to those skilled in the art from the following detailed description, wherein various configurations of the subject technology are shown and described by way of illustration. As will be realized, the subject technology is capable of other and different configurations and its several details are capable of modification in various other respects, all without departing from the scope of the subject technology. Accordingly, the drawings and detailed description are to be regarded as illustrative in nature and not as restrictive.

### BRIEF DESCRIPTION OF THE DRAWINGS

For a better understanding of the various described implementations, reference should be made to the Description of Implementations below, in conjunction with the following drawings. Like reference numerals refer to corresponding parts throughout the figures and description.

FIG. 1 depicts an example of an institutional patient care system of a healthcare organization, according to aspects of the subject technology.

FIG. 2A depicts an example of a system for determining a volume of a fluid inside a medication container, according to aspects of the subject technology.

FIG. 2B shows another example implementation of the system of FIG. 2A in which multiple separated electronic tags are utilized to monitor a volume of a fluid in a container, according to aspects of the subject technology.

FIG. 3 depicts an example of a medication container having two RFID tags for determining a volume of a fluid inside the medication container, according to aspects of the subject technology.

FIG. 4 depicts an example process for determining a volume of fluid inside a medication container, according to aspects of the subject technology.

FIG. 5 is a conceptual diagram illustrating an example electronic system 500 for determining a volume of fluid inside a medication container, according to aspects of the subject technology.

### DESCRIPTION

Reference will now be made to implementations, examples of which are illustrated in the accompanying drawings. In the following description, numerous specific details are set forth in order to provide an understanding of the various described implementations. However, it will be apparent to one of ordinary skill in the art that the various described implementations may be practiced without these specific details. In other instances, well-known methods, procedures, components, circuits, and networks have not been described in detail so as not to unnecessarily obscure aspects of the implementations.

Intravenous (IV) containers, such as infusion bags, are bags made of plastic material that contain fluids having volume of between 250 to 1000 mL. The containers are typically disposable as a result of sterile requirements. Under normal operations, some pumps run faster, though still within the tolerance of the pumps' performance specifications. For example, a pump infusing at 60 ml/hour is expected to empty a 500 mL an IV bag of medicine in a little over eight hours. If it runs 5% faster, the pump will empty a 500 mL bag approximately 20 minutes earlier than expected. In some instances, an increase pumping speed can result in air being drawn into the pump. In such a case, the pump will sound an alarm after the air has reached the air-in-line (AIL) sensor. The caregiver then may have to disconnect the set, prime it to remove the air, and then restart the infusion. Such an interruption presents a potential for introducing infections, adding time and work for the caregiver, and adding steps that can introduce errors. Thus, there is a need for methods and systems that monitor a volume of a fluid in IV containers so that timely notifications that a medication container is empty, or is about to become empty, can be provided.

The methods and systems according to aspects of the subject technology use sensing elements attached to IV infusion fluid containers (e.g., bags, bottles, etc.) that are low in cost and complexity, and do not unduly increase the cost of IV infusions. According to aspects of the subject technology, a single large radio frequency identity (RFID) tag, or a number of RFID tags are used to monitor a level of fluid in an IV container. One or more RFID tags are placed on the container, and a power level of a signal response from the RFID tags are detected. By monitoring a change in an RF power level of detected RFID signals from an RFID tag, a level of fluid in an IV container, to which the RFID tag has been affixed, is determined.

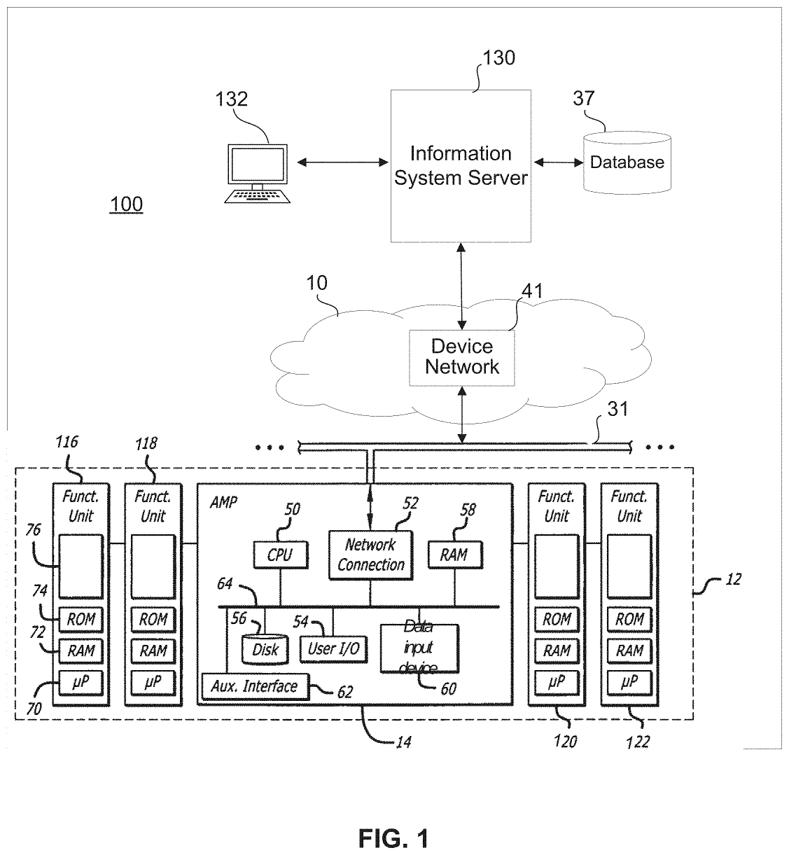

FIG. 1 depicts an example of an institutional patient care system 100 of a healthcare organization, according to aspects of the subject technology. In FIG. 1, a patient care device (or “medical device” generally) 12 is connected to a hospital network 10. The term patient care device (or “PCD”) may be used interchangeably with the term patient care unit (or “PCU”), either which may include various ancillary medical devices such as an infusion pump, a vital signs monitor, a medication dispensing device (e.g., cabinet, tote), a medication preparation device, an automated dispensing device, a module coupled with one of the aforementioned (e.g., a syringe pump module configured to attach to an infusion pump), or other similar devices. Each element 12 is connected to an internal healthcare network 10 by a transmission channel 31. Transmission channel 31 is any wired or wireless transmission channel, for example an 802.11 wireless local area network (LAN). In some implementations, network 10 also includes computer systems located in various departments throughout a hospital. For example, network 10 of FIG. 1 optionally includes computer systems associated with an admissions department, a billing department, a biomedical engineering department, a clinical laboratory, a central supply department, one or more unit station computers and/or a medical decision support system. As described further below, network 10 may include discrete subnetworks. In the depicted example, network 10 includes a device network 41 by which patient care devices 12 (and other devices) communicate in accordance with normal operations.

Additionally, institutional patient care system 100 may incorporate a separate information system server 130, the function of which will be described in more detail below. Moreover, although the information system server 130 is shown as a separate server, the functions and programming of the information system server 130 may be incorporated into another computer, if such is desired by engineers designing the institution's information system. Institutional patient care system 100 may further include one or multiple device terminals 132 for connecting and communicating with information system server 130. Device terminals 132 may include personal computers, personal data assistances, mobile devices such as laptops, tablet computers, augmented reality devices, or smartphones, configured with software for communications with information system server 130 via network 10.

Patient care device 12 comprises a system for providing patient care, such as that described in Eggers et al., which is incorporated herein by reference for that purpose. Patient care device 12 may include or incorporate pumps, physiological monitors (e.g., heart rate, blood pressure, ECG, EEG, pulse oximeter, and other patient monitors), therapy devices, and other drug delivery devices may be utilized according to the teachings set forth herein. In the depicted example, patient care device 12 comprises a control module 14, also referred to as interface unit 14, connected to one or more functional modules 116, 118, 120, 122. Interface unit 14 includes a central processing unit (CPU) 50 connected to a memory, for example, random access memory (RAM) 58, and one or more interface devices such as user interface device 54, a coded data input device 60, a network connection 52, and an auxiliary interface 62 for communicating with additional modules or devices. Interface unit 14 also, although not necessarily, includes a main non-volatile storage unit 56, such as a hard disk drive or non-volatile flash memory, for storing software and data and one or more internal buses 64 for interconnecting the aforementioned elements.

In various implementations, user interface device 54 is a touch screen for displaying information to a user and allowing a user to input information by touching defined areas of the screen. Additionally or in the alternative, user interface device 54 could include any means for displaying and inputting information, such as a monitor, a printer, a keyboard, softkeys, a mouse, a track ball and/or a light pen. Data input device 60 may be a bar code reader capable of scanning and interpreting data printed in bar coded format. Additionally or in the alternative, data input device 60 can be any device for entering coded data into a computer, such as a device(s) for reading a magnetic strips, radio-frequency identification (RFID) devices whereby digital data encoded in RFID tags or smart labels (defined below) are captured by the reader 60 via radio waves, PCMCIA smart cards, radio frequency cards, memory sticks, CDs, DVDs, or any other analog or digital storage media. Other examples of data input device 60 include a voice activation or recognition device or a portable personal data assistant (PDA). Depending upon the types of interface devices used, user interface device 54 and data input device 60 may be the same device. Although data input device 60 is shown in FIG. 1 to be disposed within interface unit 14, it is recognized that data input device 60 may be integral within pharmacy system 34 or located externally and communicating with pharmacy system 34 through an RS-232 serial interface or any other appropriate communication means. Auxiliary interface 62 may be an RS-232 communications interface, however any other means for communicating with a peripheral device such as a printer, patient monitor, infusion pump or other medical device may be used without departing from the subject technology. Additionally, data input device 60 may be a separate functional module, such as modules 116, 118, 120 and 122, and configured to communicate with controller 14, or any other system on the network, using suitable programming and communication protocols.

Network connection 52 may be a wired or wireless connection, such as by Ethernet, WiFi, BLUETOOTH, an integrated services digital network (ISDN) connection, a digital subscriber line (DSL) modem or a cable modem. Any direct or indirect network connection may be used, including, but not limited to a telephone modem, an MIB system, an RS232 interface, an auxiliary interface, an optical link, an infrared link, a radio frequency link, a microwave link or a WLANS connection or other wireless connection.

Functional modules 116, 118, 120, 122 are any devices for providing care to a patient or for monitoring patient condition. As shown in FIG. 1, at least one of functional modules 116, 118, 120, 122 may be an infusion pump module such as an intravenous infusion pump for delivering medication or other fluid to a patient. For the purposes of this discussion, functional module 116 is an infusion pump module. Each of functional modules 118, 120, 122 may be any patient treatment or monitoring device including, but not limited to, an infusion pump, a syringe pump, a PCA pump, an epidural pump, an enteral pump, a blood pressure monitor, a pulse oximeter, an EKG monitor, an EEG monitor, a heart rate monitor or an intracranial pressure monitor or the like. Functional module 118, 120 and/or 122 may be a printer, scanner, bar code reader or any other peripheral input, output or input/output device.

Each functional module 116, 118, 120, 122 communicates directly or indirectly with interface unit 14, with interface unit 14 providing overall monitoring and control of device 12. Functional modules 116, 118, 120, 122 may be connected physically and electronically in serial fashion to one or both ends of interface unit 14 as shown in FIG. 1, or as detailed in Eggers et al. However, it is recognized that there are other means for connecting functional modules with the interface unit that may be utilized without departing from the subject technology. It will also be appreciated that devices such as pumps or patient monitoring devices that provide sufficient programmability and connectivity may be capable of operating as stand-alone devices and may communicate directly with the network without connected through a separate interface unit or control unit 14. As described above, additional medical devices or peripheral devices may be connected to patient care device 12 through one or more auxiliary interfaces 62.

Each functional module 116, 118, 120, 122 may include module-specific components 76, a microprocessor 70, a volatile memory 72 and a nonvolatile memory 74 for storing information. It should be noted that while four functional modules are shown in FIG. 1, any number of devices may be connected directly or indirectly to central controller 14. The number and type of functional modules described herein are intended to be illustrative, and in no way limit the scope of the subject technology. Module-specific components 76 include any components necessary for operation of a particular module, such as a pumping mechanism for infusion pump module 116.

While each functional module may be capable of a least some level of independent operation, interface unit 14 monitors and controls overall operation of device 12. For example, as will be described in more detail below, interface unit 14 provides programming instructions to the functional modules 116, 118, 120, 122 and monitors the status of each module.

Patient care device 12 is capable of operating in several different modes, or personalities, with each personality defined by a configuration database. The configuration database may be a database 56 internal to patient care device, or an external database 37. A particular configuration database is selected based, at least in part, by patient-specific information such as patient location, age, physical characteristics, or medical characteristics. Medical characteristics include, but are not limited to, patient diagnosis, treatment prescription, medical history, medical records, patient care provider identification, physiological characteristics or psychological characteristics. As used herein, patient-specific information also includes care provider information (e.g., physician identification) or a patient care device's 10 location in the hospital or hospital computer network. Patient care information may be entered through interface device 52, 54, 60 or 62, and may originate from anywhere in network 10, such as, for example, from a pharmacy server, admissions server, laboratory server, and the like.

Medical devices incorporating aspects of the subject technology may be equipped with a Network Interface Module (NIM), allowing the medical device to participate as a node in a network. While for purposes of clarity the subject technology will be described as operating in an Ethernet network environment using the Internet Protocol (IP), it is understood that concepts of the subject technology are equally applicable in other network environments, and such environments are intended to be within the scope of the subject technology.

Data to and from the various data sources can be converted into network-compatible data with existing technology, and movement of the information between the medical device and network can be accomplished by a variety of means. For example, patient care device 12 and network 10 may communicate via automated interaction, manual interaction or a combination of both automated and manual interaction. Automated interaction may be continuous or intermittent and may occur through direct network connection 54 (as shown in FIG. 1), or through RS232 links, MIB systems, RF links such as BLUETOOTH, IR links, WLANS, digital cable systems, telephone modems or other wired or wireless communication means. Manual interaction between patient care device 12 and network 10 involves physically transferring, intermittently or periodically, data between systems using, for example, user interface device 54, coded data input device 60, bar codes, computer disks, portable data assistants, memory cards, or any other media for storing data. The communication means in various aspects is bidirectional with access to data from as many points of the distributed data sources as possible. Decision-making can occur at a variety of places within network 10. For example, and not by way of limitation, decisions can be made in HIS server 30, decision support 48, remote data server 49, hospital department or unit stations 46, or within patient care device 12 itself.

All direct communications with medical devices operating on a network in accordance with the subject technology may be performed through information system server 30, known as the remote data server (RDS). In accordance with aspects of the subject technology, network interface modules incorporated into medical devices such as, for example, infusion pumps or vital signs measurement devices, ignore all network traffic that does not originate from an authenticated RDS. The primary responsibilities of the RDS of the subject technology are to track the location and status of all networked medical devices that have NIMs, and maintain open communication.

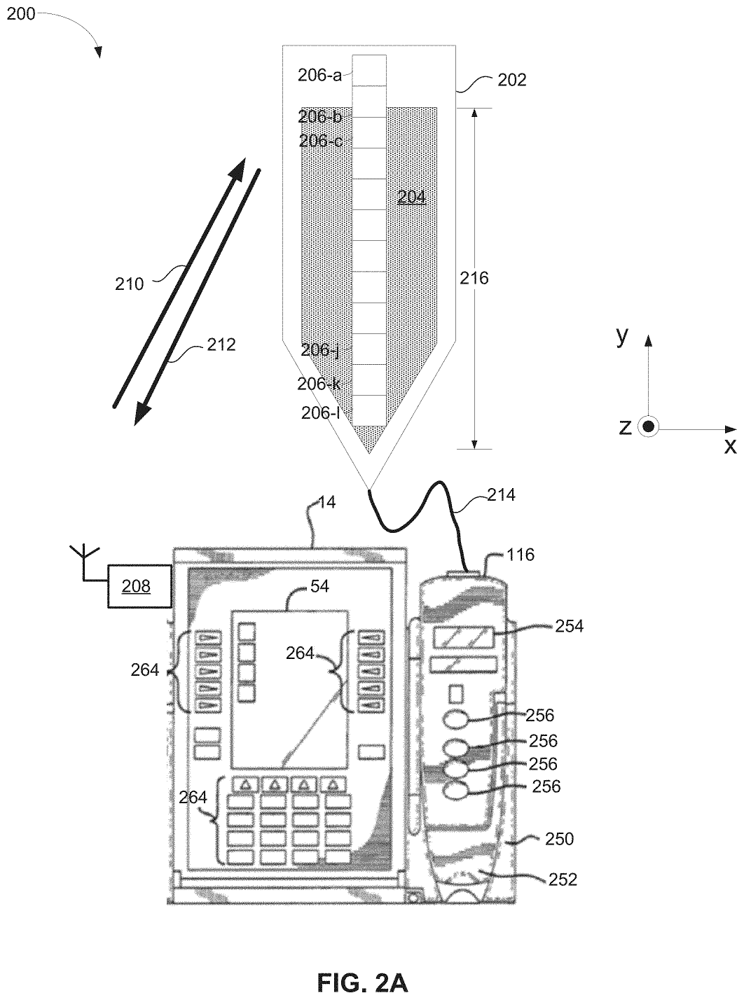

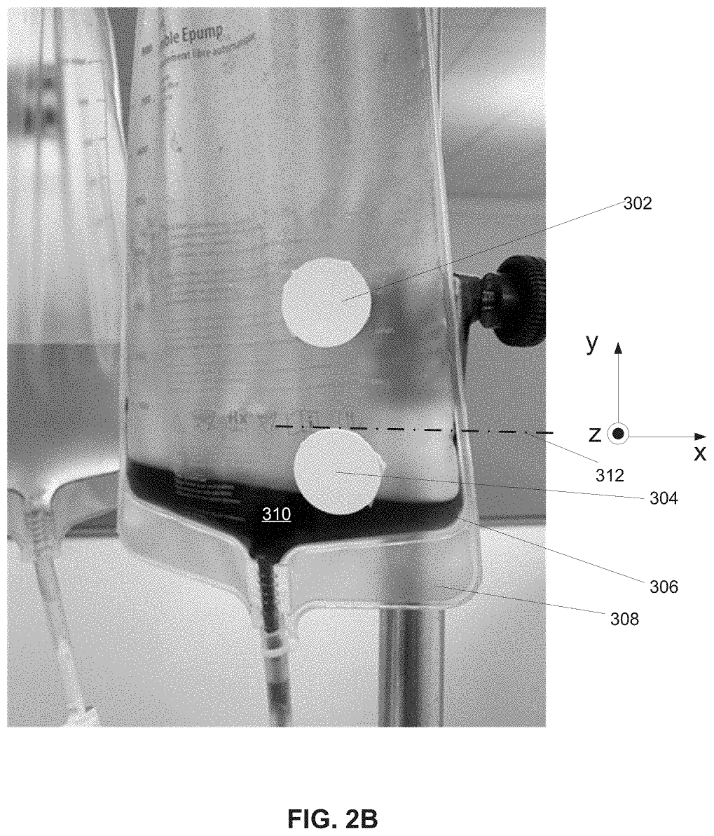

FIG. 2A shows an example system 200 that monitors a volume of a fluid in a medication container by way of a series of electronic tags affixed to the container, according to aspects of the subject technology. The medication container is, for example, an IV bag 202. The IV bag 202 is enlarged with respect to a pump 22 to illustrate aspects of the subject technology. The IV bag 202 includes a fluid 204 that is infused, using the pump 22, to a patient. A height 216 of the fluid 204 changes depending on a volume of the fluid 204 in the IV bag 202.

FIG. 2A shows ten radio frequency identify (RFID) tags 206-a, 206-b, 206-c, 206-d, 206-e, 206-f, 206-g, 206-h, 206-i, 206-k, 206-k, 206-1, affixed to an exterior of the IV bag 202. RFID tags can be manufactured in volume for very low costs, be sterilized by common sterilization methods, and can be readily applied (e.g., affixed) to the outside surface of IV container. In FIG. 2, most of the RFID tags (e.g., 206-c to 206-1) are adjacent (e.g., separated only by a layer of the IV bag 202) the fluid 204, in a z-direction (e.g., into the plane of FIG. 2). The RFID tag 206-a is adjacent air (e.g., a y-position of the RFID tag 206-a is higher than the height 216), while a portion of the RFID tag 206-b is adjacent air and a remaining portion of the RFID tag 206-b is adjacent the fluid 204 in the IV bag 202.

According to various implementations, as shown in FIG. 2A, multiple RFID tags may be placed adjacent to each other in a series to form a contiguous strip of tags (e.g., on a base strip of material which is then attached to the bag). In other implementations, different numbers of RFID tags are used. For example, in some implementations, a single RFID tag is used. In some implementations, a single RFID tag spans a portion (e.g., a quarter, a half, more than three-quarters) of a total length (along the height 216 dimension) of the IV bag 202. In some implementations, a number (e.g., two, three, four, five, six, seven, . . . or more than twenty etc.) of RFID tags are affixed to positions on the IV bags 202 that are spaced apart (e.g., not contiguous as shown in FIG. 2). In the foregoing examples, the tags may be attached to an underlying strip of material (adjacent each other or spaced apart on the material) and the material affixed to the bag 202.

In the depicted example, an infusion device control module 14 (hereinafter referred to as infusion device 14) includes an internal RFID reader 208. The internal RFID reader may include a sensor attached to or implemented in the housing of the infusion device, adjacent to a location configured to secure an IV bag. As will be described further with regard to FIG. 2B, reader 208 may be an external device. Reader 208 includes an radiofrequency (RF) source, such as a transmitter (“TX”), that emits RF radiation 210. In some implementations, the RF radiation is low frequency (LF) RF radiation, e.g., between 30-300 kHz, 120-150 kHz.

Reader 208 may include one or more transceivers or independent transmitters and receivers, for communicating with respective electronic tags affixed to the medication container 202. For the purpose of this disclosure the terms “transceiver” and/or “receiver” and/or “transmitter”, and/or “receiver/transmitter” are used interchangeably and may refer to one or multiple transceivers or one or multiple of a combination of transmitters and receivers. In some implementations, when the receiver/transmitter (TX/RX) of reader 208 is very close to the tags. In various implementations, the electronic tags are RFID tags, and may operate at a frequency of about 150 kHz. For example, in some implementations, the TX/RX unit may be placed at the pole clamp near to the IV bags for this purpose. In some implementations, when the TX/RX unit is in the pump or control module, the pump or control module (and the TX/RX unit) may be placed at about 1 m away from the IV bag. In such a configuration, RFID tags may operate in the frequency range of about 13 to 900 MHz.

The control module 14 may also include one or more input devices, such as control keys 264 or a bar code scanner (not shown) for scanning information relating to the infusion, the patient, the clinician, or other. In some implementations, the display 54 may be implemented as a touchscreen display.

The functional modules 116 includes a door 250 and a handle 252 that operates to lock the door in a closed position for operation and to unlock and open the door for access to the internal pumping and sensing mechanisms and to load administration sets for the pump. A display 254, such as an LED display, may be located in plain view on the door in some implementations and may be used to visually communicate various information relevant to the functional modules 116, such as alert indications (e.g., alarm messages). Control keys 256 exist for programming and controlling operations of the functional modules 116 as desired. In some implementations, the control keys may be omitted and be presented as interactive elements on the display 254 (e.g., touchscreen display). The functional modules 116 also includes audio alarm equipment in the form of a speaker (not shown).

In some implementations, the IV bag 202 is positioned no more than 3 feet from the infusion device 14. In some implementations, the RFID tags 206-a to 206-1 are passive RFID tags that each includes an antenna for receiving and transmitting RF signal, and a microchip (e.g., an integrated circuit that stores and processes information and modulates and demodulates RF signals). The tag information is stored in a non-volatile memory on the microchip. Passive RFID tags do not include batteries; instead, the tags use radio energy transmitted by an RF source (e.g., in the RF reader). In some implementations, RFID tags are read-only, and each RFID tag includes a factory-assigned serial number that allows the RF reader to identify specific RFID tags. As RFID tags have unique serial numbers, the RFID reader is able to discriminate among several tags that are within the range of the RFID reader and read them simultaneously.

The passive RFID tags 206-a to 206-1 respond to the RF radiation 210 or signal from the RFID reader 208 when an internal antenna of the RFID tag draws in energy from the RF radiation 210 and uses that energy to power the RFID tag's own microchip. The RFID tag's microchip generates an RF signal 212 that encodes information (e.g., specific to the RFID tag), and the signal 212 is detected by the RFID reader 208. For example, the RF signal 212 originates from the RFID tag 206-c and encodes information about the identity of the RFID tag 206-c. The RFID reader 208 is then able to decipher the source of the RF signal 212 as originating from the RFID tag 206-c.

The fluid 204 within an IV bag is typically a fluid based medication in which water is the main constituent. When an RFID tag is adjacent the fluid 204 (e.g., the RFID tag is separated only by a layer of the IV bag 202, along the z-direction, from the fluid 204), the background dielectric to the RFID tag is thus basically water. Water is a polar dielectric, which cancels out most of an incoming electric field passing through the water. Cancelling out of the incoming electric field is equivalent to the fluid absorbing the RF signal from the RFID reader 208. In some implementations, the RFID tags are placed on a side of the bag opposite RFID reader 208, such that any signal transmission between the RFID reader 208 and the RFID tags 206 passes through the IV bag, and the fluid therein. In other words, signals from the RFID reader 208 is directed through an interior space of the IV bag 202, and the one or more RFID tags 206 are disposed on the IV bag 202 opposite the interior space. In this configuration, the return RF signal from the RFID tag (e.g., when the RFID tag is only half-covered with fluid) would also pass through the fluid before the RFID reader 208 can detect it. The signal may also have to traverse through the plastic which could attenuate the signal. This configuration can be used in cases where there is no fluid to tag interaction.

When an RF signal 210 is sent out by the RFID reader 208 through the IV bag to a respective RFID tag, less energy (or no energy) is available for the internal antenna of the RFID to draw from the RF signal 210 when there is a high dielectric fluid adjacent to the RFID tag. The microchip of the RFID tag 206 in turn has less energy (or no energy) for its microchip to generate a response RF signal 212. A signal strength of the response RF signal 212 may therefore provide an indication about a level of the fluid in the IV bag.

In some implementations, one or more RFID tags are placed on a front side of the IV bag (e.g., in front of the fluid). The presence of the high dielectric fluid behind the RFID tag detunes a characteristic frequency (e.g., resonance frequency) of the internal antenna of the RFID tag, rendering the internal antenna of the RFID tag less able (or unable) to draw energy from the RF signal 210 emitted by the RFID reader 208. As a result, less energy (or no energy) is available for the microchip of the RFID tag to generate a return RF signal 212 when there is a high dielectric fluid adjacent to the RFID tag.

When the fluid level 216 has dropped below the position (e.g., along the y-direction) of a particular RFID tag (e.g., RFID tag 206-a), the internal antenna of that RFID tag (e.g., RFID 206-a) is able to draw in most or all of the RF energy from the signal 210 because air does not cancel out the electric field of the RF signal 210, unlike water. Thus, the return signal 212 generated by the RFID tag 206-a is stronger when the RFID tag is no longer adjacent to water. As a result, the system 200 is able to sense the presence of fluid 204 in the vicinity of a RFID tag 206 based on a controlled degradation of the RFID tag's power characteristics (e.g., in the return RF signal 212).

In some implementations, the signal strength of the return RF signal 212 is monitored by Received Signal Strength Indication (RSSI) power levels. RSSI is a measurement of the power present in a received radio signal. The signal is then correlated to the level of the fluid in the IV container. The amount of a tag's surface area covered by fluid may be proportional to the tags perceived signal strength. Accordingly, when a tag in the form of a strip is placed on a side of an IV bag in a linear direction corresponding to a height of the fluid within the bag when the bag is hung in position, the signal strength of the tag may be indicative of a height of the fluid within the bag.

In some implementations, the correlation between a surface area of a tag (or a strip of tags) that is adjacent to a fluid, the signal strength, and an amount of fluid within the bag may be determined based on a calibration process. During calibration, the power characteristics of one or more RFID tags is measured as a function of the volume of a specific type of fluid in the IV bag, prior to an infusion process for that specific type of fluid. In some implementations, when a single RFID tag is used over a portion of the IV bag 202, the calibration process includes generating a look-up table, which associates a received power in the return RF signal 212 detected by the RF reader 210 with a height (and thus volume) of the fluid 204 present in the IV bag. For a single RFID tag, the fluid present in the medication container detunes the antenna, resulting in an RF signal having a reduced signal strength from the RFID tag. As the fluid level drops in the medication container, less of the RFID tag is adjacent to the fluid, resulting in an RF signal that increases in signal strength.

In some implementations, the system 200 includes an RFID reader 208 that senses the RSSI power to make a determination about the fluid level in a medication container. The RFID reader 208 communicates with the infusion pump to provide fluid level information including, in some implementations, a signal strength value indicative of a volume of fluid remaining, and a processor 50 of the infusion pump performs a lookup to determine the amount of fluid remaining in the IV bag. In some implementations, the RFID reader 208 receives the RF signal, and the processing of that received signal is done by some processors (in the pump, control unit, or reader). In some implementations, the processing may be done in the PCU pump control unit. In some implementations, where the processing of the received signal is done may be dependent on where the RFID TX/RX unit is placed. The processing may be performed by the reader and an indication of the fluid level transmitted by the reader to the pump. In some implementations, the reader may periodically read the tags and the pump may query the reader for a current reading, or instruct the reader to take a reading and return the resulting value. If necessary, the processor generates an alert that the medication container is empty, prior to the infusion line emptying and drawing air into the system. In some implementations, the infusion pump includes software to convert RFID tag signals to actionable messages to a caregiver (e.g., change the medication container).

An example way of configuring the pump to administer a medication to a patient according to aspects of the subject technology (hereinafter referred to as the work process) includes starting a program to initialize the IV-bag volume monitoring system (“IV-BVMS”). In some implementations, the IV-BVMS includes the system 200.

The work process includes entering information about the infusion process. Information that is entered may include the medication to be administered, the IV bag's filled volume (e.g., the total volume of fluids in the IV bag at the start of the infusion process). Some medications may have stronger dielectric properties than others. In this regard, different signal strengths may be associated with different medications, and thus a lookup table may correlate different signal strengths for different medications with the same fluid level. Some infusion pumps may have multiple channels that permit multiple medications to be delivered to the same patient. For infusion pumps having multiple channels, information about pump association, for example, which channel of the pump is associated with which IV bag, can also be entered in this part of the work process. The work process can include reading the RFID tag on the IV bag to verify the information entered so far in the work process. The last steps of the work process include connecting the IV lines (e.g., the administration set) to the pump before starting the IV infusion process.

During the infusion process, the IV-BVMS, which was earlier initialized, detects signals from the RFID tags disposed on the IV bag at periodic intervals. In some implementations, signals from the RFID tags are detected periodically every 1-5 minutes (e.g., RFID tags are read at one minute intervals, RFID tags are read at two minute intervals, RFID tags are read at three minute intervals, RFID tags are read at four minute intervals, RFID tags are read at five minute intervals, etc.). The length of the periodic interval for detection may depend on a flow rate of the infusion and the volume of fluids in the IV bag. More frequent detections may be made for high flow rate. In some implementations, the detection intervals change (e.g. shorten) as the infusion progresses. At the start of the infusion process, when there is a relative large amount of fluids remaining the IV bag, detections of the IV bag volume may be made less frequently. As the volume of the fluids in the IV bag decreases, more frequent detections of the volume of the fluid remaining in the bag may be made in order to provide a timely warning to a clinician when the IV bag would soon become empty.

The detection process of the RFID tag includes the internal RFID reader 208 sending the RF signal 210 out, and the RFID reader 208 receiving a return signal 212 from one or more RFID tags 206-a . . . 206-1. In some implementations, a single RF signal would be sent out to trigger responses from most (e.g., all) of the RFID tags on the IV bag 202, for example, all the RFID tags disposed along the entire height of the IV bag 202 during the operation. In some implementations, the RFID tags may include configuration information, and the RFID reader 208 may be used during set up of the infusion to scan the configuration information prior to the administration of the medication. The configuration information may include an identification of the respective tag and its location relative to other tags on the bag. In some implementations, a master configuration RFID tag (or barcode) may be placed on the IV bag, which includes configuration information (e.g., identifiers and placement) for all of the tags on the bag. The processor 50 of the infusion pump is configured to receive the configuration tag, and determine the appropriate lookup table to use to determine the amount of fluid in the bag during the infusion (e.g., based on the type of medication, number of tags, placement of tags, identifiers, and the like).

In some implementations, the RFID reader 208 is configured to detect/read multiple signals (e.g., from each of the 10 tags) and process the signals sequentially. In some implementations, the signal is processed into one of two binary states (e.g., either on or off); the RFID tags that are on (e.g., when there is no fluid in the IV bag behind the RFID tags) may transmit an ID number. The ID number may correlate to the (e.g., y-direction) position on the IV bag to indicate the level of fluid present in the bag. In some implementations, once a strong signal (e.g., RF signal having a signal strength above the threshold) is obtained from a first tag (“tag-1”), the tag(s) above (e.g., tags positioned higher along the y-direction) are presumed to also emit a high signal, allowing the detection to be limited to tags below (e.g., lower along the y-direction) the first tag. In some implementations, the RFID reader 208 is configured to monitor a trend of the signal, and accounts for cases where a strong signal is detected only momentarily due to a temporary deviation.

In some implementations, no RF signal is received from the RFID tag when there is fluid adjacent the RFID tag. In such implementations, the RF reader detects two binary states—either a RF signal is received, indicating that there is no fluid at the height of the RFID tag, or no RF signal is received, indicating that there is fluid at the height of the RFID tag.

In some implementations, RSSI power provides a quantitative measurement of the amount of returned RF power. In such implementations, when the RF reader detects a returned RF signal that is equal or greater than a predetermined threshold, the fluid level in the medication container is deemed to be lower than the height corresponding to the position of the RFID tag and/or its surface area. In some implementations, the predetermined threshold is obtained by calibrating the system while a medication container is draining. In such implementations, at the beginning of the infusion process, when the fluid level is higher than the position of the RFID tag, the RF reader either does not register any RF signal or registers only a low level of RF signal. As the infusion process progresses, the detected RF signal starts to increase as the fluid level drains to a vicinity of the RFID tag. For applications in which an early warning is desired, the predetermined threshold can be set to a lower magnitude. In general, the infusion process starts with the RFID reader not detecting much (or any RF signals), and as the infusion process progresses, the RFID reader detects a maximum value when the fluid level falls below the tag.

In some implementations, the IV-BVMS calculates a volume of the fluids remaining. The calculation can be done at a processor 50 of the pump. Results of the measurements detected at the RFID reader 208 is transmitted to a server, and a processor at the server performs calculations relating to the amount of remaining fluids in the IV container.

For a first RFID tag for which no RF signal or RF signals below a threshold is obtained, the IV bag would be deemed to have fluid levels that covers at least half of the vertical/height dimension (e.g., y-direction) of that tag. In some implementations, when the RFID reader 208 determines that a second tag just above the first tag (for which no signal or signals below the threshold is obtained) emits a strong signal, the processor sets the fluid level of the IV bag to be somewhere between the first tag and the second tag.

The IV-BVMS displays the volume remaining it has calculated based on the signals detected by the RFID reader 208, as a confirmation of the normal operation of the infusion pump. In some implementations, the IV-BVMS transmits the information via a wireless communication (e.g., WiFi) connection to a clinician (e.g., a caregiver at a nursing station) for monitoring.

The IV-BVMS also compares the calculated infused volume (based on the volume of the fluid remaining as detected by the RFID reader 208) with an expected volume infused (that is based on a time and flow rate of the pump). If the volume infused differs from the expected volume infused by a set percentage (e.g., 5%, 7%, 10%, 15%, 20%) the IV-BVMS issues an alert for a clinician to check the infusion process.

When the volume remaining in the IV bag is below a set limit (e.g., less than 10%, less than 5%, less than 2%, etc.), an alert is displayed or sounded, or sent to a computing device 132 associated with the clinician (e.g., a mobile device or smartphone) to indicate that the bag is nearly empty. The set limit is determined based on the total volume and flow rate of the infusion, to ensure adequate time is provided to replace the bag.

When the RFID tags indicate that all volume is expelled from the bag (e.g, when all the RFID-tags are returning a signal having a power that is above the threshold value), the infusion pump will be stopped and an alarm will notify the caregiver that the IV bag is completely empty.

FIG. 2B shows another example implementation of the system of FIG. 2A in which multiple separated electronic tags 302 and 304 are utilized to monitor a volume of a fluid in a container, according to aspects of the subject technology. In the depicted example, the electronic tags are not contiguous to each other but are placed at two separate locations on an IV bag 308. A fluid level 306 of a fluid 310 in the IV bag 308 is close to the tag 304, and a lower RF signal is received from the tag 304. In contrast, because the tag is 302 is adjacent air in the z-direction, a larger RF signal is detected from the tag 302. As a result, the IV-BVMS determines/calculates that the volume of the fluid 310 remaining in the IV bag 308 is less than a height associated with the location of the tag 302.

As the fluid 310 drains through the height dimension (e.g., from a height 312 to the height 306), the RF signal 312 returned from the tag 306 increases. The changing strength of the RF signal indicates the fluid level is changing at the heights covered by the tag 304.

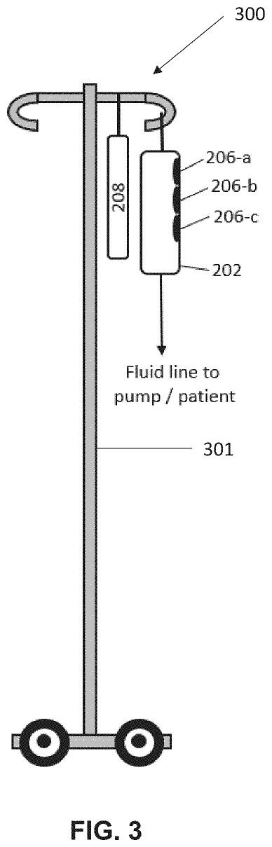

FIG. 3 shows depicts an example intravenous (IV) pole 300 with an example medication container 202 and an example reader 208 hanging therefrom, according to aspects of the subject technology. In the depicted example, the disclosed reader 208 is hung from an upper appendage of vertical mast 301 of pole 300, adjacent to an IV bag 202 which is also hanging from pole 300. For example, reader 308 may hang from an arm at or near the top of vertical mast, or from an anchor for an appendage from which the fluid container hangs. Tags 206-a to 206-1 are affixed to a side of bag 202 and reader 208 and bag 202 positioned such that transmission from reader 208 to tags 206-a to 206-1 occurs through bag 202 and its contents. In the depicted example, bag 202 is hung on an outer appendage of pole 300 while reader 202 is hung from an inner appendage between the pole and bag 202 in a manner such that the electronic tags are on a side of the bag opposite the reader.

Reader 208 may be attached via a pole mount to a vertical mast of pole 300, as shown. In some implementations, reader 208 may be integrated into vertical mast 301. For example, the vertical mast 301 may be configured with the circuitry of reader 208 (including a processor) embedded therein. In such embodiments, the radio transmitters of reader 208 (e.g., the circuitry) may be positioned near a top of the vertical mast 301 at a location corresponding to a predicted height of container 202, such that the radio transmitters align or substantially align with electronic tags 206-a to 206-1 when container 202 is attached to pole 300.

Reader 208 may communicate with control module 14 or a functional module 116, 118, 120, 122 such as the described infusion pump via a wired (e.g., USB) or wireless (e.g., WiFi, Bluetooth, etc.) connection. In some implementations, reader 208 may connect to a (separate) monitoring device configured to consume and report (e.g., via a display screen) the information provided by reader 208. The reader 208 may communicate and/or receive power from a source integrated into or with the pole. For example, power and data connection cables may be routed through the vertical mast 301 to pump and/or a power outlet. Accordingly, reader 208 may include a wired connection with the pump. In some implementations, the reader may include a mounting element to hang the reader from the top of an infusion pole at a deterministic position relative to the medication container. In implementations in which reader 208 hangs from a top of pole 300 by a mounting element, the attachment or anchor may provide the power or data connection cables (e.g., which may feed through the vertical mast).

Reader 208 may include one or more RF devices such as one or more transceivers or a combination of one or more transmitters and receivers. In this regard, each RF device may provide an RF transmitting source and a RF receiving source. According to various implementations, reader 208 may transmits (via the RF transmitting source) an RF signal toward a plurality of RFID tags 206-a to 206-1 disposed on a side of a medication container 202 associated with an infusion device administering a medication from the medication container. The infusion device may be a control unit 14 or a functional module 116, 118, 120, 122 such as the described infusion pump. The RFID tags are placed on the side of the medication container opposite a side of the medication container nearest the RF transmitting source such that the RF signal passes through the medication container before interacting with the RFID tags. Reader 208 then detects (via the RF receiving source) a signal strength of returned RF signals from the RFID tags.

According to various implementations, each of the returned RF signals include an identifier identifying a respective RFID tag. Reader 208 determines, based on at least one of the returned identifiers, a threshold signal level associated with detecting a fluid within the medication container. For example, the RFID tags may be associated with a particular medication, and the returned identifier may be used by the reader 208 to lookup transmission characteristics of the medication fluid. The lookup may be performed with a lookup table accessible to the reader (e.g., in a memory of the reader) or by way of the reader querying a remote server or database using the identifier and obtaining the characteristic(s) in response to the query from the server or database.

Reader 208 then determines a volume of the fluid within the medication container based comparing the signal strength of each returned RF signal with the determined threshold signal level. In this regard, the tags may be placed on the medication container 202 in a predetermined order. For example, each tag may be positioned on a strip of material in a predetermined order and that order maintained in the database (or lookup table). The order may be determined by the reader (or other device determining the volume based on the readings) based on at least one of the predetermine identifiers. The order may be used to determine the volume by identifying which of the RFID tags corresponds to a signal that indicates no fluid is present. If there are four tags and positions one and two indicate no fluid is present, and tags three and four indicate that fluid is present, it may be presumed that the fluid is at a level corresponding to tag three (e.g., half full if tag three is at a location associated with the volume being half full). Accordingly, reader 208 may determine a first signal strength of a first returned RF signal satisfies the threshold signal level while a second signal strength of a second returned RF signal does not satisfy the threshold signal level, and then the volume of the fluid may be determined based on the predetermined order for placement of the plurality of RFID tags and a location, within the predetermined order, of the RFID tag providing a returned RF signal having a signal strength that does not satisfy the threshold signal level.

In some implementations, reader 208 includes a display screen, and provides a representation of the fluid level on the display screen. In some implementations, reader 208 provides an electronic indication of the volume to the control unit 14 or a functional module of the device for display by control unit or the functional module.

With reference to FIGS. 2 and 3, as a safety precaution, in some implementations, a background signal may be received from the IV bag throughout an infusion process. For example, a reference RFID tag associated with the IV bag may be positioned at a location on the IV bag sufficiently far away from any fluid. As a result, the reference RFID tag on the IV bag always returns a signal (containing information about the identity of the IV bag) during the infusion process, regardless of the fluid level in the IV bag. In this way, an absence of a return signal from a particular RFID tag is not a result of the RFID signal 210 not reaching the RFID tag.

Various clinicians can make use of the IV-BVMS system. In some implementations, the RFID tag is affixed onto the IV container by a pharmacist or a prescribing doctor. To further improve performance of the IV-BVMS system, specialized RFID tags can be used. In some implementations, the antenna of the RFID tag is designed to radiate the return RF signal 212 in a specific direction (e.g., toward the RF reader 208). In this way, the RF reader 208 may be able to detect smaller RF signals emitted by the RFID tag.

The IV-BVMS system is not limited to monitoring the use of a single IV bag at any particular time. In some implementations, the system is used to monitor more than one IV containers. For example, levels of secondary fluids from additional IV bags are monitored. In general, the IV-BVMS system is able to measure fluid levels of multiple IV bags at the same time. In some implementations, each bag is associated with a unique reference ID tag number, allowing fluid levels of multiple IV bags to be measured at the same time. For example, the clinician can identify the bag and reference the ID tag to a particular pump channel. The subject technology also allows an IV bag/medication to be associated to the pump channel as a way to reduce associational errors.

In some implementations, the processor 50 also calculates a volume of a fluid in the medication container based on a height of the fluid in the medication container. Based on the results of the calculation, the IV-BVMS system can provide an indication that the height of the fluid in the medication container is below a minimum height (e.g., a minimum height associated with the medication container emptying within a short period of time). For example, the IV-BVMS may sound an alarm to notify a clinician when the height of the fluid in the medication container is below the minimum height.

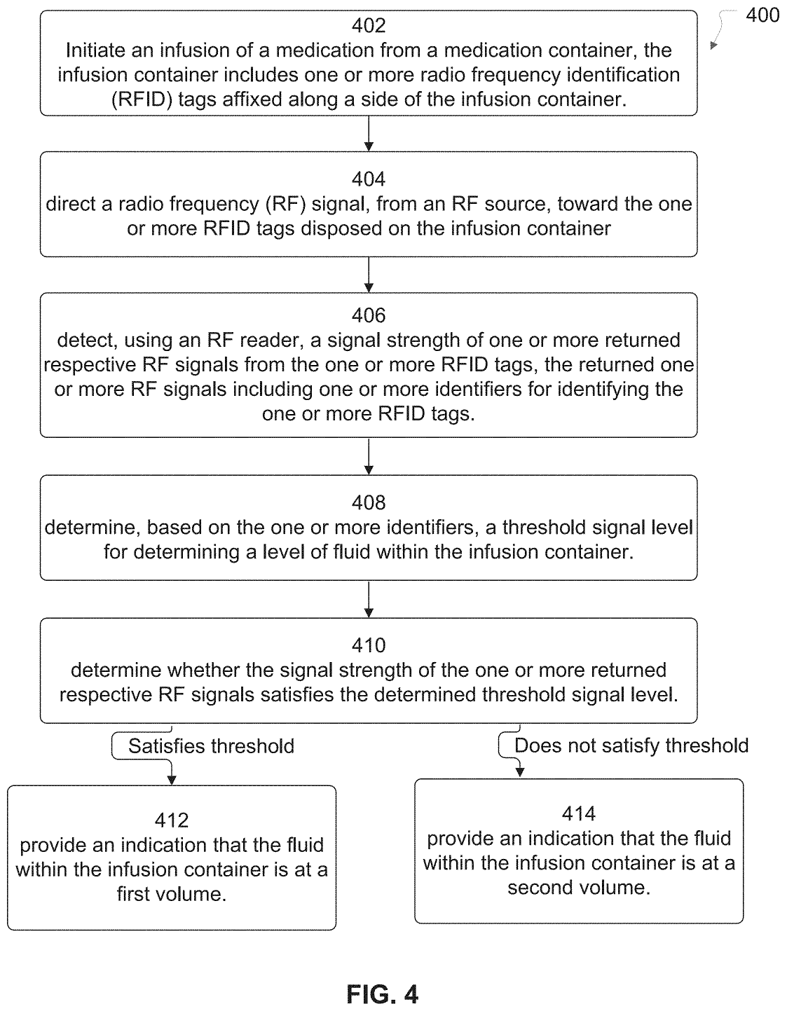

FIG. 4 depicts an example method for determining a volume of fluid in the medication container, according to aspects of the subject technology. For explanatory purposes, the various blocks of example process 400 are described herein with reference to FIGS. 1-3, and the components and/or processes described herein. The one or more of the blocks of process 400 may be implemented, for example, by one or more computing devices. In some implementations, one or more of the blocks may be implemented apart from other blocks, and by one or more different processors or devices. Further for explanatory purposes, the blocks of example process 400 are described as occurring in serial, or linearly. However, multiple blocks of example process 40 may occur in parallel. In addition, the blocks of example process 400 need not be performed in the order shown and/or one or more of the blocks of example process 400 need not be performed.

In the depicted example, an infusion of a medication from a medication container is initiated (402). The medication container includes one or more radio frequency identification (RFID) tags affixed along a side of the medication container. The IV-BVMS system causes an RF signal from an RF source to be directed toward one or more RFID tags disposed on a medication container (404). The IV-BVMS system uses an RF reader to detect a signal strength of one or more returned respective RF signals from the one or more RFID tags, the returned one or more RF signals including one or more identifiers for identifying the one or more RFID tags (406). The IV-BVMS system determines a threshold signal level for determining a level of fluid within the medication container based on the one or more identifiers (408). The IV-BVMS system determines whether the signal strength of the one or more returned respective RF signals satisfies the determined threshold signal level (410). In accordance with a determination that the signal strength of the returned RF signal satisfies the threshold signal level, the IV-BVMS system provides an indication that the fluid within the medication container is at a first volume (412). In accordance with a determination that the signal strength does not satisfy the threshold signal level, the IV-BVMS system provides an indication that the fluid within the medication container is at a second volume (414). In one aspect, a method of determining a volume of a fluid in a medication container, the method includes initiating an infusion of a medication from a medication container. The medication container includes one or more radio frequency identification (RFID) tags affixed along a side of the medication container. The method includes directing a radio frequency (RF) signal, from an RF source, toward the one or more RFID tags disposed on the medication container. The method also includes detecting, using an RF reader, a signal strength of one or more returned respective RF signals from the one or more RFID tags, the returned one or more RF signals including one or more identifiers for identifying the one or more RFID tags. The method includes determining, based on the one or more identifiers, a threshold signal level for determining a level of fluid within the medication container, and determining whether the signal strength of the one or more returned respective RF signals satisfies the determined threshold signal level. In accordance with a determination that the signal strength of the returned RF signal satisfies the threshold signal level, the method includes providing an indication that the fluid within the medication container is at a first volume; and in accordance with a determination that the signal strength does not satisfy the threshold signal level, the method includes providing an indication that the fluid within the medication container is at a second volume.

In some implementations, the method also includes calculating the volume of fluid in the medication container based on the signal strength and a number and location of the one or more RFID tags on the medication container. In some implementations, the method also includes generating an alarm when the fluid in the medication container is below a predetermined minimum volume. In some implementations, determining whether the signal strength of the one or more returned respective RF signals satisfies the determined threshold signal level is performed by a processor of a server system.

In some implementations, a determination that the returned RF signal does not satisfy the threshold signal level includes not detecting any returned RF signal from the RFID tag. In some implementations, the method includes determining the volume of the fluid in the medication container based on a look-up table that stores a correspondence between RFID tag identifiers and a respective volume of the fluid within the medication container. In some implementations, the method includes identifying a medication of the medication container based on information provided by a respective RFID tag affixed to the medication container.

In some implementations, directing the RF signal includes directing the RF signal through an interior space of the medication container, and the one or more RFID tags are disposed on the medication container opposite the interior space. In some implementations, the medication container includes an IV bag. In some implementations, the method includes checking a volume of the fluid remaining in the medication container against an expected volume infused. In some implementations, a plurality of RFID tags are affixed along a side of the medication container, the method further includes directing multiple RF signals to the plurality of RFID tags, and receiving a response from a portion of the plurality of RFID tags; and determining a volume of the fluid within the medication container based on a number of responses received from the RFID tags.

In some implementations, the medication container includes a first container and a second container, the first container including one or more first RFID tags and the second container including one or more second RFID tags, the method further includes determining a volume of a fluid in the first container based on RF signals received from the first RFID tags; and determining a volume of a fluid in the second container based on RF signals received from the second RFID tags.

In some implementations, the RFID tag has a dimension that spans more than half the height of the medication container, and a magnitude of the returned RF signal indicates a level of fluid in the medication container.

In some implementations, the method includes using a look-up table to convert the magnitude of the returned RF signal to a volume of the fluid in the medication container. In some implementations, the look-up table is obtained by calibrating the RFID tag with known amounts of fluid in the medication container. In some implementations, the first RFID tag is affixed at a position on the medication container associated with a lowest level of fluid in the medication container, below which the medication container is empty. In some implementations, the method further includes causing a pump to which the medication container is connected to stop infusing and notify a clinician when the medication container is empty.

In some implementations, the RFID tag is affixed at a position of the medication container associated with the medication container becoming empty in less than a predetermined time at a particular flow rate of the medication container, the method further includes determining, based on a strength of the RF signal returned from the first RFID tag and a current flow rate of the infusion of the medication, that the medication container will become empty in less than the predetermined time; and generating an alert indicating that the medication container will become empty in less than the predetermined time.

In some implementations, the method further includes calculating a volume delivered from the medication container based on the returned RF signal from the one or more RFID tags changing from a signal that is below the threshold signal level to a signal that is above the threshold signal level.

In some implementations, the method further includes comparing the volume delivered with an expected volume infused and sounding an alarm when a difference between the volume delivered and the expected volume infused is greater than a threshold. In some embodiments, the threshold is selected by a clinician. In some implementations, the threshold is greater than 5% (e.g., 6%, 7%, 8%, 10%, 15%, etc.). In some implementations, the threshold is less than 5% (e.g., 4%, 3%, 2%, 1%, 0.5%, etc.). In some implementations, the threshold is about 5% (e.g., between 4.5% to 5.5%). In some implementations, detecting the returned RF signal from the RFID tag comprises periodically detecting the returned RF signal throughout an infusion.

Many of the above-described method 400, and related features and applications, may also be implemented as software processes that are specified as a set of instructions recorded on a computer readable storage medium (also referred to as computer readable medium), and may be executed automatically (e.g., without user intervention). When these instructions are executed by one or more processing unit(s) (e.g., one or more processors, cores of processors, or other processing units), they cause the processing unit(s) to perform the actions indicated in the instructions. Examples of computer readable media include, but are not limited to, CD-ROMs, flash drives, RAM chips, hard drives, EPROMs, etc. The computer readable media does not include carrier waves and electronic signals passing wirelessly or over wired connections.

The term “software” is meant to include, where appropriate, firmware residing in read-only memory or applications stored in magnetic storage, which can be read into memory for processing by a processor. Also, in some implementations, multiple software aspects of the subject disclosure can be implemented as sub-parts of a larger program while remaining distinct software aspects of the subject disclosure. In some implementations, multiple software aspects can also be implemented as separate programs. Finally, any combination of separate programs that together implement a software aspect described here is within the scope of the subject disclosure. In some implementations, the software programs, when installed to operate on one or more electronic systems, define one or more specific machine implementations that execute and perform the operations of the software programs.

A computer program (also known as a program, software, software application, script, or code) can be written in any form of programming language, including compiled or interpreted languages, declarative or procedural languages, and it can be deployed in any form, including as a stand-alone program or as a module, component, subroutine, object, or other unit suitable for use in a computing environment. A computer program may, but need not, correspond to a file in a file system. A program can be stored in a portion of a file that holds other programs or data (e.g., one or more scripts stored in a markup language document), in a single file dedicated to the program in question, or in multiple coordinated files (e.g., files that store one or more modules, sub programs, or portions of code). A computer program can be deployed to be executed on one computer or on multiple computers that are located at one site or distributed across multiple sites and interconnected by a communication network.

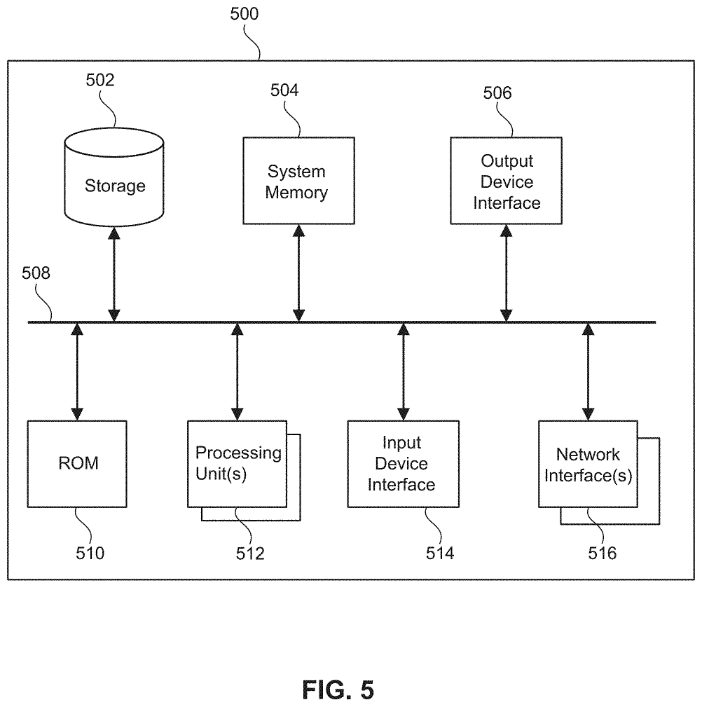

FIG. 5 is a conceptual diagram illustrating an example electronic system 500 for the automatically determining a volume of fluid in the medication container, according to aspects of the subject technology. Electronic system 500 may be a computing device for execution of software associated with one or more portions or steps of process 500, or components and processes provided by FIGS. 1-3, including but not limited to server 130, computing hardware within patient care device 12, or terminal device 132. Electronic system 500 may be representative, in combination with the disclosure regarding FIGS. 1-4. In this regard, electronic system 500 may be a personal computer or a mobile device such as a smartphone, tablet computer, laptop, PDA, an augmented reality device, a wearable such as a watch or band or glasses, or combination thereof, or other touch screen or television with one or more processors embedded therein or coupled thereto, or any other sort of computer-related electronic device having network connectivity.

Electronic system 500 may include various types of computer readable media and interfaces for various other types of computer readable media. In the depicted example, electronic system 500 includes a bus 508, processing unit(s) 512, a system memory 504, a read-only memory (ROM) 510, a permanent storage device 502, an input device interface 514, an output device interface 506, and one or more network interfaces 516. In some implementations, electronic system 500 may include or be integrated with other computing devices or circuitry for operation of the various components and processes previously described.

Bus 508 collectively represents all system, peripheral, and chipset buses that communicatively connect the numerous internal devices of electronic system 500. For instance, bus 508 communicatively connects processing unit(s) 512 with ROM 510, system memory 504, and permanent storage device 502.

From these various memory units, processing unit(s) 512 retrieves instructions to execute and data to process in order to execute the processes of the subject disclosure. The processing unit(s) can be a single processor or a multi-core processor in different implementations.

ROM 510 stores static data and instructions that are needed by processing unit(s) 512 and other modules of the electronic system. Permanent storage device 502, on the other hand, is a read-and-write memory device. This device is a non-volatile memory unit that stores instructions and data even when electronic system 500 is off. Some implementations of the subject disclosure use a mass-storage device (such as a magnetic or optical disk and its corresponding disk drive) as permanent storage device 502.

Other implementations use a removable storage device (such as a floppy disk, flash drive, and its corresponding disk drive) as permanent storage device 502. Like permanent storage device 502, system memory 504 is a read-and-write memory device. However, unlike storage device 502, system memory 504 is a volatile read-and-write memory, such a random access memory. System memory 504 stores some of the instructions and data that the processor needs at runtime. In some implementations, the processes of the subject disclosure are stored in system memory 504, permanent storage device 502, and/or ROM 510. From these various memory units, processing unit(s) 512 retrieves instructions to execute and data to process in order to execute the processes of some implementations.

Bus 508 also connects to input and output device interfaces 514 and 506. Input device interface 514 enables the user to communicate information and select commands to the electronic system. Input devices used with input device interface 514 include, e.g., alphanumeric keyboards and pointing devices (also called “cursor control devices”). Output device interfaces 506 enables, e.g., the display of images generated by the electronic system 500. Output devices used with output device interface 506 include, e.g., printers and display devices, such as cathode ray tubes (CRT) or liquid crystal displays (LCD). Some implementations include devices such as a touchscreen that functions as both input and output devices.

Also, as shown in FIG. 5, bus 508 also couples electronic system 500 to a network (not shown) through network interfaces 516. Network interfaces 516 may include, e.g., a wireless access point (e.g., Bluetooth or WiFi) or radio circuitry for connecting to a wireless access point. Network interfaces 516 may also include hardware (e.g., Ethernet hardware) for connecting the computer to a part of a network of computers such as a local area network (“LAN”), a wide area network (“WAN”), wireless LAN, or an Intranet, or a network of networks, such as the Internet. Any or all components of electronic system 500 can be used in conjunction with the subject disclosure.

These functions described above can be implemented in computer software, firmware or hardware. The techniques can be implemented using one or more computer program products. Programmable processors and computers can be included in or packaged as mobile devices. The processes and logic flows can be performed by one or more programmable processors and by one or more programmable logic circuitry. General and special purpose computing devices and storage devices can be interconnected through communication networks.

Some implementations include electronic components, such as microprocessors, storage and memory that store computer program instructions in a machine-readable or computer-readable medium (also referred to as computer-readable storage media, machine-readable media, or machine-readable storage media). Some examples of such computer-readable media include RAM, ROM, read-only compact discs (CD-ROM), recordable compact discs (CD-R), rewritable compact discs (CD-RW), read-only digital versatile discs (e.g., DVD-ROM, dual-layer DVD-ROM), a variety of recordable/rewritable DVDs (e.g., DVD-RAM, DVD-RW, DVD+RW, etc.), flash memory (e.g., SD cards, mini-SD cards, micro-SD cards, etc.), magnetic and/or solid state hard drives, read-only and recordable Blu-Ray® discs, ultra density optical discs, any other optical or magnetic media, and floppy disks. The computer-readable media can store a computer program that is executable by at least one processing unit and includes sets of instructions for performing various operations. Examples of computer programs or computer code include machine code, such as is produced by a compiler, and files including higher-level code that are executed by a computer, an electronic component, or a microprocessor using an interpreter.

While the above discussion primarily refers to microprocessor or multi-core processors that execute software, some implementations are performed by one or more integrated circuits, such as application specific integrated circuits (ASICs) or field programmable gate arrays (FPGAs). In some implementations, such integrated circuits execute instructions that are stored on the circuit itself.

As used in this specification and any claims of this application, the terms “computer”, “server”, “processor”, and “memory” all refer to electronic or other technological devices. These terms exclude people or groups of people. For the purposes of the specification, the terms display or displaying means displaying on an electronic device. As used in this specification and any claims of this application, the terms “computer readable medium” and “computer readable media” are entirely restricted to tangible, physical objects that store information in a form that is readable by a computer. These terms exclude any wireless signals, wired download signals, and any other ephemeral signals.

To provide for interaction with a user, implementations of the subject matter described in this specification can be implemented on a computer having a display device, e.g., a CRT (cathode ray tube) or LCD (liquid crystal display) monitor, for displaying information to the user and a keyboard and a pointing device, e.g., a mouse or a trackball, by which the user can provide input to the computer. Other kinds of devices can be used to provide for interaction with a user as well; e.g., feedback provided to the user can be any form of sensory feedback, e.g., visual feedback, auditory feedback, or tactile feedback; and input from the user can be received in any form, including acoustic, speech, or tactile input. In addition, a computer can interact with a user by sending documents to and receiving documents from a device that is used by the user; e.g., by sending web pages to a web browser on a user's client device in response to requests received from the web browser.

Embodiments of the subject matter described in this specification can be implemented in a computing system that includes a back end component, e.g., as a data server, or that includes a middleware component, e.g., an application server, or that includes a front end component, e.g., a client computer having a graphical user interface or a Web browser through which a user can interact with an implementation of the subject matter described in this specification, or any combination of one or more such back end, middleware, or front end components. The components of the system can be interconnected by any form or medium of digital data communication, e.g., a communication network. Examples of communication networks include a local area network (“LAN”) and a wide area network (“WAN”), an inter-network (e.g., the Internet), and peer-to-peer networks (e.g., ad hoc peer-to-peer networks).

The computing system can include clients and servers. A client and server are generally remote from each other and may interact through a communication network. The relationship of client and server arises by virtue of computer programs running on the respective computers and having a client-server relationship to each other. In some implementations, a server transmits data (e.g., an HTML page) to a client device (e.g., for purposes of displaying data to and receiving user input from a user interacting with the client device). Data generated at the client device (e.g., a result of the user interaction) can be received from the client device at the server.

Those of skill in the art would appreciate that the various illustrative blocks, modules, elements, components, methods, and algorithms described herein may be implemented as electronic hardware, computer software, or combinations of both. To illustrate this interchangeability of hardware and software, various illustrative blocks, modules, elements, components, methods, and algorithms have been described above generally in terms of their functionality. Whether such functionality is implemented as hardware or software depends upon the particular application and design constraints imposed on the overall system. The described functionality may be implemented in varying ways for each particular application. Various components and blocks may be arranged differently (e.g., arranged in a different order, or partitioned in a different way) all without departing from the scope of the subject technology.

It is understood that the specific order or hierarchy of steps in the processes disclosed is an illustration of example approaches. Based upon design preferences, it is understood that the specific order or hierarchy of steps in the processes may be rearranged. Some of the steps may be performed simultaneously. The accompanying method claims present elements of the various steps in a sample order, and are not meant to be limited to the specific order or hierarchy presented.

The previous description is provided to enable any person skilled in the art to practice the various aspects described herein. The previous description provides various examples of the subject technology, and the subject technology is not limited to these examples. Various modifications to these aspects will be readily apparent to those skilled in the art, and the generic principles defined herein may be applied to other aspects. Thus, the claims are not intended to be limited to the aspects shown herein, but is to be accorded the full scope consistent with the language claims, wherein reference to an element in the singular is not intended to mean “one and only one” unless specifically so stated, but rather “one or more.” Unless specifically stated otherwise, the term “some” refers to one or more. Pronouns in the masculine (e.g., his) include the feminine and neuter gender (e.g., her and its) and vice versa. Headings and subheadings, if any, are used for convenience only and do not limit the invention described herein.

The term website, as used herein, may include any aspect of a website, including one or more web pages, one or more servers used to host or store web related content, etc. Accordingly, the term website may be used interchangeably with the terms web page and server. The predicate words “configured to”, “operable to”, and “programmed to” do not imply any particular tangible or intangible modification of a subject, but, rather, are intended to be used interchangeably. For example, a processor configured to monitor and control an operation or a component may also mean the processor being programmed to monitor and control the operation or the processor being operable to monitor and control the operation. Likewise, a processor configured to execute code can be construed as a processor programmed to execute code or operable to execute code.

The term automatic, as used herein, may include performance by a computer or machine without user intervention; for example, by instructions responsive to a predicate action by the computer or machine or other initiation mechanism. The word “example” is used herein to mean “serving as an example or illustration.” Any aspect or design described herein as “example” is not necessarily to be construed as preferred or advantageous over other aspects or designs.

A phrase such as an “aspect” does not imply that such aspect is essential to the subject technology or that such aspect applies to all configurations of the subject technology. A disclosure relating to an aspect may apply to all configurations, or one or more configurations. An aspect may provide one or more examples. A phrase such as an aspect may refer to one or more aspects and vice versa. A phrase such as an “embodiment” does not imply that such embodiment is essential to the subject technology or that such embodiment applies to all configurations of the subject technology. A disclosure relating to an embodiment may apply to all implementations, or one or more implementations. An embodiment may provide one or more examples. A phrase such as an “embodiment” may refer to one or more embodiments and vice versa. A phrase such as a “configuration” does not imply that such configuration is essential to the subject technology or that such configuration applies to all configurations of the subject technology. A disclosure relating to a configuration may apply to all configurations, or one or more configurations. A configuration may provide one or more examples. A phrase such as a “configuration” may refer to one or more configurations and vice versa.

What is claimed is:

1. An monitoring device for monitoring a volume of a medication container, comprising:
one or more radio frequency (RF) devices providing an RF transmitting source and a RF receiving source;
one or more processors; and
a non-transitory memory device having instructions thereon that, when executed by the one or more processors, cause the monitoring device to perform operations comprising:
transmitting, via the RF transmitting source, an RF signal toward a plurality of RF identification (RFID) tags disposed on a side of a medication container associated with an infusion device administering a medication from the medication container, wherein the side of the medication container is opposite a side of the medication container nearest the RF transmitting source such that the RF signal passes through the medication container before interacting with the RFID tags;
detecting, via the RF receiving source, a signal strength of returned RF signals from the RFID tags, each of the returned RF signals including an identifier identifying a respective RFID tag;
determining, based on at least one of the returned identifiers, a threshold signal level associated with detecting a fluid within the medication container;
determining a volume of the fluid within the medication container based comparing the signal strength of each returned RF signal with the determined threshold signal level; and
providing an electronic indication of the volume.

2. The monitoring device of claim 1, wherein determining the volume of the fluid comprises:
determining a first signal strength of a first returned RF signal satisfies the threshold signal level while a second signal strength of a second returned RF signal does not satisfy the threshold signal level;
determining a predetermined order for placement of first and second RFID tags of the the RFID tags based on at least one of the returned identifiers;
determining the volume of the fluid based on the predetermined order for placement of the plurality of RFID tags and a location, within the predetermined order, of the RFID tag providing a returned RF signal having a signal strength that does not satisfy the threshold signal level.

3. The monitoring device of claim 1, wherein providing the electronic indication of the volume comprises:
providing the electronic indication to the infusion device for display at the infusion device.

4. The monitoring device of claim 1, further comprising:
a display screen,
wherein the operations further comprise:
displaying a representation of the electronic indication on the display screen.

5. A method of determining a volume of a fluid in a medication container, comprising:
initiating an infusion of a medication from a medication container, wherein the medication container comprises one or more radio frequency identification (RFID) tags affixed along a side of the medication container;
directing a radio frequency (RF) signal, from an RF source, toward the one or more RFID tags disposed on the medication container;
detecting, using an RF reader, a signal strength of one or more returned respective RF signals from the one or more RFID tags, the returned one or more RF signals including one or more identifiers for identifying the one or more RFID tags;
determining, based on the one or more identifiers, a threshold signal level for determining a level of fluid within the medication container;
determining whether the signal strength of the one or more returned respective RF signals satisfies the determined threshold signal level;
in accordance with a determination that the signal strength of the returned RF signal satisfies the threshold signal level, providing an indication that the fluid within the medication container is at a first volume; and
in accordance with a determination that the signal strength does not satisfy the threshold signal level, providing an indication that the fluid within the medication container is at a second volume.

6. The method of claim 5, further comprising:
calculating the volume of fluid in the medication container based on the signal strength and a number and location of the one or more RFID tags on the medication container.

7. The method of claim 5, further comprising:
generating an alarm when the fluid in the medication container is below a predetermined minimum volume.

8. The method of claim 5, wherein determining whether the signal strength of the one or more returned respective RF signals satisfies the determined threshold signal level is performed by a processor of a server system.

9. The method of claim 5, wherein a determination that the returned RF signal does not satisfy the threshold signal level comprises not detecting any returned RF signal from the RFID tag.

10. The method of claim 5, further comprising determining the volume of the fluid in the medication container based on a look-up table that stores a correspondence between RFID tag identifiers and a respective volume of the fluid within the medication container.

11. The method of claim 5, further comprising identifying a medication of the medication container based on information provided by a respective RFID tag affixed to the medication container.

12. The method of claim 5, wherein directing the RF signal comprises directing the RF signal through an interior space of the medication container, and the one or more RFID tags are disposed on the medication container opposite the interior space.

13. The method of claim 5, further comprising:
checking a volume of the fluid remaining in the medication container against an expected volume infused.

14. The method of claim 5, wherein a plurality of RFID tags are affixed along a side of the medication container, the method further comprising:
directing multiple RF signals to the plurality of RFID tags, and receiving a response from a portion of the plurality of RFID tags; and
determining a volume of the fluid within the medication container based on a number of responses received from the RFID tags.

15. The method of claim 5, further comprising using a look-up table to convert the magnitude of the returned RF signal to a volume of the fluid in the medication container.

16. The method of claim 5, wherein a first RFID tag is affixed at a position on the medication container associated with a lowest level of fluid in the medication container, below which the medication container is empty.

17. The method of claim 16, further comprising causing a pump to which the medication container is connected to stop infusing and transmit a notification to a clinician device when the medication container is empty.

18. The method of claim 5, wherein a first RFID tag is affixed at a position of the medication container associated with the medication container becoming empty in less than a predetermined time at a particular flow rate of the medication container, the method further comprising:
determining, based on a strength of the RF signal returned from the first RFID tag and a current flow rate of the infusion of the medication, that the medication container will become empty in less than the predetermined time; and
generating an alert indicating that the medication container will become empty in less than the predetermined time.

19. The method of claim 5, further comprising calculating a volume delivered from the medication container based on the returned RF signal from the one or more RFID tags changing from a signal that is below the threshold signal level to a signal that is above the threshold signal level.

20. The method of claim 19, further comprising comparing the volume delivered with an expected volume infused and sounding an alarm when a difference between the volume delivered and the expected volume infused is greater than a threshold.
